(19) United States
(12) Patent Application Publication
(10) Pub. No.: US 2025/0387131 A1
(43) Pub. Date: Dec. 25, 2025

(54) RETRIEVAL DEVICES, SYSTEMS, AND RELATED ASSEMBLIES AND METHODS

(71) Applicant: Boston Scientific Scimed, Inc., Maple Grove, MN (US)

(72) Inventors: Julian FUENTES CASTRO, La Unión (CR); Jose Roberto SOTO GUZMAN, Alajuela (CR); Cristopher OVIEDO MIRANDA, Alajuela (CR); Laura Emily SABBAN, Norton, MA (US); Juan Pablo ORTIZ GARCIA, Heredia (CR); Gian Franco LOO FUCHS, San Jose (CR); Ana Lucia PEREZ SABORÍO, San Jose (CR)

(73) Assignee: Boston Scientific Scimed, Inc., Maple Grove, MN (US)

(21) Appl. No.: 19/240,643

(22) Filed: Jun. 17, 2025

Related U.S. Application Data
(60) Provisional application No. 63/662,130, filed on Jun. 20, 2024.

(51) Int. Cl. A61B 17/221; A61B 17/00; A61B 17/22

(52) U.S. Cl. CPC A61B 17/221; A61B 2017/00818; A61B 2017/22038; A61B 2017/2215

(57) ABSTRACT

Medical devices, systems, and related methods for capturing objects within a subject (e.g., patient) are described. The medical device includes a sheath, a shaft, and a retrieval device coupled to a distal end of the shaft. At least a portion of the shaft is movably disposed within the sheath. The retrieval device includes a plurality of wires coupled to the distal end of the shaft, and a distal tip coupled to distal ends of the plurality of wires. The distal tip includes a body, and a channel extending through the body that is configured to receive a medical instrument therein.

### CROSS-REFERENCE TO RELATED APPLICATIONS

This application claims the benefit of priority to U.S. Provisional Application No. 63/662, 130, filed on Jun. 20, 2024, which is incorporated by reference herein in its entirety.

### TECHNICAL FIELD

Various aspects of the disclosure relate generally to retrieval devices, systems, and related assemblies and methods. Examples of the disclosure relate to retrieval devices, systems, and related assemblies and methods, for example, concerning a medical instrument coupled with an expandable basket or expandable element, among other aspects.

### BACKGROUND

Advancements in medical devices, systems, and related methods, have enabled users to perform increasingly complex medical procedures. One challenge in the field of medical procedures is associated with extracting objects from a patient, such as a biliary stone from a bile duct of the patient. These procedures often require multiple instruments to extract the object, which may cause various procedural issues and/or increase the risk of injury to the patient. In some aspects, the object(s) are captured with an expandable retrieval basket. For example, the expandable retrieval basket may be guided or otherwise delivered to a treatment site (e.g., a bile duct) via a guidewire or one or more other medical tools that are associated with medical procedures for extracting object(s) from the patient. In these aspects, it can be difficult to safely and/or securely couple the expandable retrieval basket to the guidewire or medical tool(s). It would be useful to improve medical extraction devices, systems, and related methods to mitigate the risk of procedural issues and/or injury to the patient during medical procedures that involve extracting objects.

### SUMMARY

Examples of this disclosure relate to, among other things, medical devices such as retrieval devices, medical systems, and related assemblies and methods.

According to one example, a medical device may include a sheath, a shaft, and a retrieval device coupled to a distal end of the shaft. The shaft may include at least a portion movably disposed within the sheath. The retrieval device may include a plurality of wires coupled to the distal end of the shaft, and a distal tip coupled to distal ends of the plurality of wires. The distal tip may include a body, and a channel extending through the body between a first opening and a second opening, in which the channel of the distal tip is configured to receive the medical instrument therein. The retrieval device may be movable between at least a closed state and an open state, may be configured to radially compress into the closed state when positioned within the sheath, and may be configured to radially expand into the open state when positioned outside of the sheath.

Any medical device or medical system described herein may include any of the following features. The distal tip may include a slot extending through the body along a longitudinal axis of the distal tip, in which the slot is coupled to distal ends of the plurality of wires therein. The medical device may include a tube coupled to distal ends of the plurality of wires, and in which the slot of the distal tip is coupled to the tube received therein. The plurality of wires may be coupled within the tube via an adhesive, a crimp, a weld, or a combination thereof. The tube may be coupled within the slot of the distal tip via an adhesive, a crimp, a weld, or a combination thereof. The tube may include a body having a lumen extending therein along a longitudinal axis between a proximal end and a distal end of the distal tip, and at least one opening extending through the body into the lumen, in which at least one of the plurality of wires is received through the at least one opening of the tube and secured within the lumen of the body. At least one opening of the tube may include a plurality of openings extending through the body into the lumen and positioned circumferentially about the longitudinal axis, in which each of the plurality of openings is configured to receive at least one of the plurality of wires therein.

The plurality of wires may radially expand into the open state to define a cavity configured to capture an object therein. The plurality of wires may be coupled to the slot of the distal tip via an adhesive, a crimp, a weld, or a combination thereof. The second opening of the channel may be positioned distal of the slot relative to the longitudinal axis of the distal tip. The channel of the distal tip may extend through the body along an axis that is oriented at an acute angle relative to the longitudinal axis of the distal tip. The body of the distal tip may include a lip, in which the lip is formed by a radially outward extension positioned proximal of a proximal end of the slot of the distal tip. A distal end of the sheath may include a distal surface configured to contact the lip of the distal tip in response to proximal translation of the retrieval device relative to the sheath. The medical instrument may include a guidewire slidably positioned in the channel of the distal tip. The distal tip may include a distally tapering outer surface at a distal end of the distal tip.

According to another example, a medical device may include a sheath, a shaft, and a retrieval device coupled to a distal end of the shaft. The retrieval device may include a plurality of wires, a distal tip, and a guide insert. The distal tip may include a body and a slot, in which the slot extends through the body along a longitudinal axis of the distal tip, and in which the slot of the distal tip is configured to receive distal ends of the plurality of wires therein. The guide insert may be disposed in a distal portion of the slot of the distal tip, in which the guide insert is configured to receive a portion of a guidewire. The retrieval device may be movable between at least a closed state and an open state, may be configured to radially compress into the closed state when positioned within the sheath, and may be configured to radially expand into the open state when positioned outside of the sheath,

Any medical device or medical system described herein may include any of the following features. The guide insert may include a body and a guide slot. The guide slot may extend through the body between a first aperture and a second aperture, in which the guide slot is configured to receive the portion of the guidewire. The distal tip may include a body having a lumen extending therein along a longitudinal axis between a proximal end and a distal end of the distal tip, and a plurality of openings extending through the body into the lumen and positioned circumferentially about the longitudinal axis. At least one of the plurality of openings may be configured to receive at least one set of the plurality of wires therein. The first aperture of the guide insert may align with at least one of the plurality of openings of the distal tip to receive the portion of the guidewire therein.

According to another example, a medical system may include a sheath, a shaft, a retrieval device coupled to a distal end of the shaft, and a guidewire movably disposed in the retrieval device. The retrieval device may include a plurality of wires coupled to the distal end of the shaft, and a distal tip coupled to distal ends of the plurality of wires. The distal tip may include a body, a slot extending through the body along a longitudinal axis of the distal tip, and a channel extending through the body along an axis that is oriented at a non-zero angle relative to the longitudinal axis. The slot of the distal tip may be coupled to distal ends of the plurality of wires therein. The guidewire may be movably disposed in the channel of the distal tip. The retrieval device may movable between at least a closed state and an open state, may be configured to radially compress into the closed state when positioned within the sheath, and may be configured to radially expand into the open state when positioned outside of the sheath.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIGS. 1A and 1B show a perspective view and longitudinal cross-sectional view of various portions of a medical device, according to aspects of the disclosure.

FIG. 2 shows a longitudinal cross-sectional view of a portion of another medical device, according to aspects of the disclosure.

FIG. 3 shows a perspective view of a portion of yet another medical device and an exemplary guidewire, according to aspects of the disclosure.

FIG. 4 shows a perspective view of a portion of another medical device, according to aspects of the disclosure.

### DETAILED DESCRIPTION

Examples of the disclosure include retrieval devices, systems, and related assemblies and methods, for example, for coupling expandable members of a retrieval device of an extraction assembly or medical device. For example, the expandable members may be selectively expandable for collecting and/or removing one or more objects from a target treatment site within a patient. Various aspects of this disclosure may help, for example, to slidably couple a portion of the retrieval device to a guidewire or other medical device.

As used herein, the term “distal” refers to a portion farthest away from a user when introducing a device into a patient and the term “proximal” refers to a portion closest to the user when placing the device into the subject. The terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to retrieval devices, systems, and related assemblies and methods for performing various medical procedures and/or treating portions of the biliary duct, large intestine, small intestine, cecum, esophagus, any other portion of the gastrointestinal tract, and/or any other suitable patient anatomy (collectively referred to herein as a “target treatment site”). Various examples described herein include single-use or disposable medical devices, although some embodiments may include one or more reusable components of the device. Reference will now be made in detail to examples of the disclosure described above and illustrated in the accompanying drawings. Wherever possible, the same reference numbers will be used throughout the drawings to refer to the same or like parts

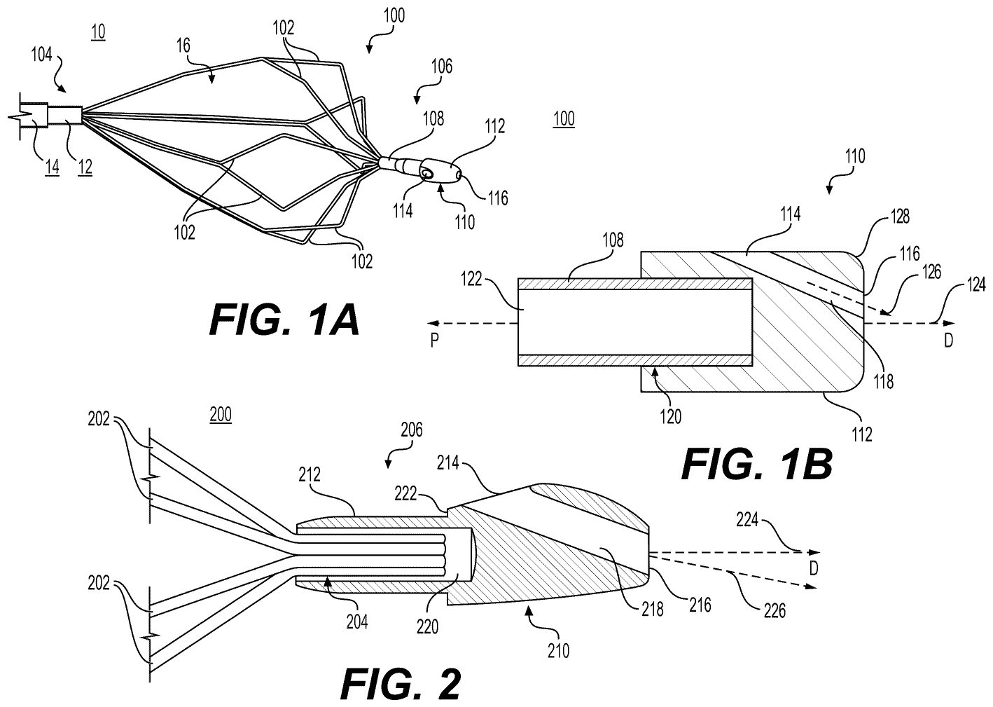

FIGS. 1A-1B illustrate a medical device 10, according to aspects of the disclosure. As shown, the medical device 10 may include a retrieval device 100 coupled to a distal end of a shaft 12. The retrieval device 100 may include a plurality of wires 102 extending between a proximal end 104 and a distal end 106 of the retrieval device 100, and may be movable between at least a closed state and an open state. The plurality of wires 102 may include proximal ends coupled to a distal end of a shaft 12 at the proximal end 104 of the retrieval device 100, and distal ends coupled to a coupler or tube, for example, a hypotube 108, at the distal end 106 of the retrieval device 100.

In some aspects, the retrieval device 100 may radially compress into the closed state and radially expand into the open state. For example, the retrieval device 100 may radially compress into the closed state when positioned within a sheath 14 of the medical device 10, and radially expand when positioned outside of the sheath 14. As shown, the shaft 12 may be at least partially disposed in the sheath 14. The shaft 12 and the sheath 14 may move (e.g., translate and/or rotate) relative to each other. For example, the distal end of the shaft 12 may translate distally or proximally relative to the sheath 14, which may cause the retrieval device 100 to translate relative to the sheath 14. From the closed state, the retrieval device 100 may translate distally through the sheath 14 and radially expand (e.g., into the open state) when the retrieval device 100 extends distal of the distal end of the sheath 14. In the open state, the retrieval device 100 may define a cavity 16 for capturing one or more objects therein (e.g., a biliary stone from a bile duct).

In some aspects, the medical device 10 may be delivered through various insertion devices or delivery systems to a target treatment site. For example, although not shown, the medical device 10 may be delivered through working channel or other lumen of an endoscope, duodenoscope, gastroscope, colonoscope, ureteroscope, bronchoscope, and/or various other insertion devices or delivery systems to the target treatment site. The medical device 10 may include a handle (not shown) having one or more ports configured to receive and/or control one or more medical instruments therein and/or one or more actuators. For example, the handle of the medical device 10 may be configured to move the retrieval device 100 between the closed state and the open state during medical procedures, for example, to capture the object(s) therein.

As shown, the plurality of wires 102 may radially expand to define the cavity 16 of the retrieval device 100 (e.g., in the open state for capturing objects therein). The plurality of wires 102 may include, for example, wire filament splitting into multiple wires in a substantially bulbous shape and converging at ends thereof. For instance, the plurality of wires 102 may include two, three, four, five, six, seven, eight or more wires which collectively form the substantially bulbous shape. The plurality of wires 102 may include one or more flexible materials (e.g., Nitinol) that allow the retrieval device 100 to self-expand into the open state (e.g., when positioned outside of or distal to the sheath 14). Additionally, or alternatively, one or more portions of the retrieval device 100 may be formed as or otherwise include a net and/or mesh that facilitates retrieval of smaller fragments of material. In other implementations, the retrieval device 100 may be configured to expand manually into the open state.

FIG. 1B shows a longitudinal cross-section view of the retrieval device 100 of FIG. 1A. In some aspects, the hypotube 108 is shaped and dimensioned to receive distal ends of the plurality of wires 102 therein, and the distal ends of wires 102 may be secured within the hypotube 108 using one or more fixation or coupling processes (e.g., crimping, welding, soldering, adhesive bonding, etc.). As shown, the hypotube 108 of the retrieval device 100 includes an inner surface defining a lumen 122 extending therein along a longitudinal axis between the proximal end and distal end of the hypotube 108. The hypotube 108 includes an opening (e.g., proximal opening) that extends into the lumen 122 and that is configured to receive one or more medical instruments therein. For example, as shown, the proximal opening of the hypotube 108 is shaped and dimensioned to receive distal ends of the plurality of wires 102 therein, and the distal ends of wires 102 may be secured to the inner surface defining the lumen 122 of the hypotube 108 using one or more fixation or coupling processes.

In some aspects, it may be useful to couple the retrieval device 100 to one or more medical instruments, for example, to guide the distal end 106 of the retrieval device 100 into a body lumen of a patient during medical procedures. The retrieval device 100 may include one or more components configured to couple the hypotube 108 to the medical instrument(s). For example, the retrieval device 100 may include a distal tip 110 having a first portion (e.g., proximal portion) coupled to a first medical instrument (e.g., hypotube 108), and a second portion (e.g., distal portion) coupled to a second medical instrument (e.g., guidewire). The distal tip 110 may be integrally formed with the hypotube 108, or may be attached to the hypotube 108 by, for example, an adhesive, a snap-fit connection, threaded connection, or the like. The distal tip 110 may include an attachment feature (not shown), such as an internal thread or latch, configured for securing the distal tip 110 to and/or radially around the hypotube 108. For example, the distal tip 110 may include an internal feature configured to engage and couple to an external feature of the hypotube 108, such as internal threading configured to threadably engage external thread. It should be understood that the attachment feature may, additionally or alternatively, include other mechanisms to secure the hypotube 108 and the distal tip 110, such as a latch or lock.

As shown, the distal tip 110 of the retrieval device 100 may include a body 112 extending between a proximal surface and a distal surface of the distal tip 110. The body 112 may include an inner surface defining a slot 120 extending therein along a longitudinal axis of the retrieval device 100 from the proximal surface toward the distal surface. The slot 120 of the distal tip 110 may include an opening (e.g., proximal opening) shaped and dimensioned to receive one or more medical instruments therein (e.g., the hypotube 108). For example, the proximal opening of the slot 120 may be configured to receive a distal end of the hypotube 108 therein, such that the hypotube 108 may be received within and distally advanced through a portion of the body 112 of the distal tip 110. In one example, the hypotube 108 is advanced distally or otherwise positioned along a length of the slot 120 and secured within the distal tip 110 using one or more fixation or coupling processes, such as adhesive bonding. In other examples, the slot 120 may include an attachment feature configured for securing the distal tip 110 to and/or radially around the hypotube 108, such as internal thread configured to threadably engage external thread.

As further shown, the body 112 of the distal tip 110 may include an inner surface defining a channel 118 extending therein between a first opening 114 and a second opening 116 formed in the body 112. The first opening 114 of the channel 118 may be positioned on, and extend through, a side surface that extends between the proximal surface and distal surface of the body 112 of the distal tip 110. The second opening 116 of the channel 118 may be positioned on, and extend through, the distal surface of the body 112 of the distal tip 110. In some aspects, the first opening 114 and the second opening 116 of the channel 118 are shaped and dimensioned to receive a guidewire therein, such that the guidewire may be advanced through the channel 118. For example, the guidewire (not shown) may be advanced distally or otherwise slidably positioned through the first opening 114 into the channel 118 and through the second opening 116.

In some aspects, the channel 118 of the distal tip 110 may be oriented at a non-zero angle (e.g., an acute angle) relative to the slot 120 of the distal tip 110. The slot 120 may extend through the body 112 along a first axis 124 of the retrieval device 100, and the channel 118 may extend through the body 112 along a second axis 126 of the retrieval device 100 different than the first axis 124. For example, the slot 120 may extend along the longitudinal axis of the retrieval device 100 (e.g., first axis 124), and the channel 118 may extend along another axis (e.g., second axis 126) at an angle relative to the longitudinal axis. The medical instrument(s) may be advanced distally or otherwise slidably positioned through the channel 118 at the angle relative to the longitudinal axis of the retrieval device 100. In one example, a distal portion of a guidewire (not shown) may be advanced distally or otherwise slidably positioned through the channel 118 of the distal tip 110, such that the guidewire is advanced distally or otherwise slidably positioned through the second opening 116 along the second axis 126 and positioned distal of the channel 118 at an angle relative to the longitudinal axis of the retrieval device 100 (e.g., first axis 124).

In some aspects, the distal tip 110 may be atraumatic or have atraumatic surfaces, which may help to allow for atraumatic insertion and/or movement of the retrieval device 100 into a body lumen of a patient. For instance, in some examples, the distal tip 110 has an outer surface 128 shaped and dimensioned to contact tissue of the patient without causing damage to said tissue, such as a curved or distally tapering outer surface. The distal tip 110 may have variable dimensions at different portions along a length thereof, for example a distally tapering surface shaped and dimensioned for atraumatic contact with tissue of the patient. The distal tip 110 may include one or more materials (e.g., ceramics, plastics, metals, alloys, etc.) and formed using one or more manufacture processes (e.g., additive manufacturing, reductive manufacturing, machining, molding, stamping, etc.).

In some aspects, it may be useful to provide a medical device that combines various aspects of the retrieval device 100 in a single component. For example, it may be useful to provide a retrieval device combining aspects of the distal tip 110 and aspects of the hypotube 108 into a single component.

FIG. 2 shows another retrieval device 200, according to aspects of the disclosure. Aspects of the retrieval device 200 may be similar to the retrieval device 100 (FIGS. 1A-1B), the details of which are omitted for brevity. For instance, a medical device may include a shaft (not shown) and the retrieval device 200 coupled to a distal end of the shaft. The retrieval device 200 may be movable between at least a closed state and an open state. The retrieval device 200 may radially compress into the closed state and radially expand into the open state. For example, the retrieval device 200 may radially compress into the closed state when positioned within a sheath of the medical device, and radially expand when positioned outside of the sheath. The shaft may be at least partially disposed in the sheath, such that the shaft and the sheath may move (e.g., translate and/or rotate) relative to each other. In the open state, the retrieval device 200 may define a cavity for capturing one or more objects therein (e.g., biliary stone from bile duct).

The retrieval device 200 may include a plurality of wires 202. The plurality of wires 202 may include proximal ends coupled to the distal end of the shaft positioned at a proximal end of the retrieval device 200 (not shown). The plurality of wires include distal ends 204 coupled to a distal tip 210 positioned at a distal end 206 of the retrieval device 200. The distal tip 210 may include a body 212 extending along a longitudinal axis between a proximal surface and a distal surface. The body 212 of the distal tip 210 may include an inner surface defining a slot 220 extending therein from the proximal surface toward the distal surface. The slot 220 includes at least one opening (e.g., a proximal opening) that is shaped and dimensioned to receive the distal ends 204 of the plurality of wires 202 therein. In one example, the distal ends 204 of the plurality of wires 202 are advanced distally or otherwise slidably positioned through the slot 220 of the distal tip 210, and at least one of the distal ends 204 are secured within the distal tip 210 using one or more fixation or coupling processes (e.g., crimping, welding, soldering, adhesive bonding, etc.). As shown, the proximal opening of the slot 220 extends distally through the proximal surface of the body 212 toward the distal surface along the longitudinal axis of the distal tip 210. However, it should be understood that the distal tip 210 may alternatively or additionally have different slots extending therein. For example, the distal tip 210 may include a plurality of openings extending distally through the proximal surface of the body 212 into a plurality of slots, such that each slot may receive one or more of the plurality of wires 202.

In some aspects, it may be useful to couple the retrieval device 200 to one or more medical instruments, for example, to guide the distal end 206 of the retrieval device 200 into a body lumen of a patient during medical procedures. As shown, the body 212 of the distal tip 210 may include an inner surface defining a channel 218 extending therein between a first opening 214 and a second opening 216 formed in the body 212. In one example, the channel 218 is shaped and dimensioned to receive a guidewire, such that the guidewire may be advanced distally or otherwise slidably positioned through the first opening 214 into the channel 218 and through the second opening 216. The channel 218 of the distal tip 210 may be oriented at a non-zero angle (e.g., an acute angle) relative to the slot 220 of the distal tip 210. The slot 220 may extend along a first axis 224 of the distal tip 210, and the channel 218 may extend along a second axis 226 of the distal tip 210 at the non-zero angle relative to the first axis 224. In one example, the slot 220 extends along a longitudinal axis of the distal tip 210 (e.g., first axis 224), and the channel 218 extends along another axis (e.g., second axis 226) at an acute angle relative to the longitudinal axis of the distal tip 110. The medical instrument(s) may be advanced distally or otherwise slidably positioned through the first opening 214 into the channel 218 and through the second opening 216 of the distal tip 210, such that a distal portion of the medical instrument(s) is oriented at the acute angle to the longitudinal axis of the distal tip 210.

In some aspects, the distal tip 210 may include one or more surfaces that are shaped and dimensioned to prevent proximal translation of the retrieval device 200, for example, relative to a distal end of a sheath. As shown, the body 212 of the distal tip 210 may have a variable cross-section along a length thereof relative to the longitudinal axis of the distal tip 210 which may define a lip 222 positioned on the outer surface of the body 212. The lip 222 may be formed by a radially outward extension that is positioned proximal of the first opening 214 of the channel 218 relative to the longitudinal axis of the distal tip 210. The lip 222 may be shaped and dimensioned to contact or form a stop surface for the distal end of the sheath at the proximal end of the retrieval device 200 (not shown). For example, proximal retraction of the retrieval device 200 relative to the sheath may drive the lip 222 into contact with one or more surfaces at the distal end of the sheath. The lip 222 may have a complementary shape that mates with the surface(s) of the sheath to prevent the distal tip 210 from translating proximal of the distal end of the sheath. For example, the lip 222 is formed by the radially outward extension which complements the shape of a distal surface at a distal end of the sheath.

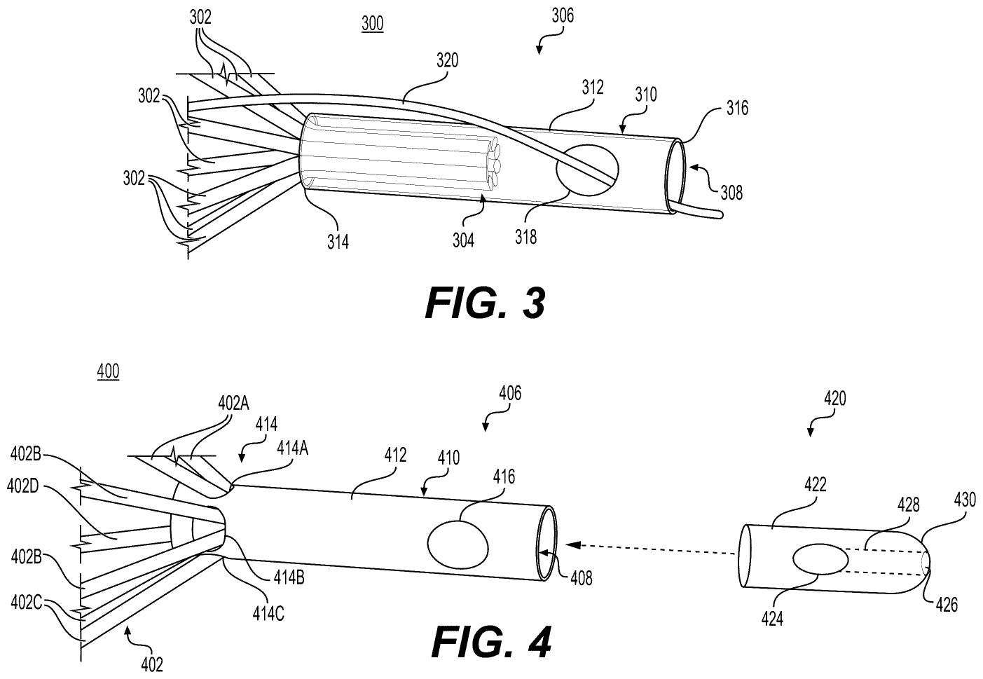

FIG. 3 shows a retrieval device 300 of another medical device, according to aspects of the disclosure. Aspects of the retrieval device 300 may be similar to the retrieval device 100 (FIGS. 1A-1B) and/or the retrieval device 200 (FIG. 2), the details of which are omitted for brevity. For instance, a medical device may include a shaft (not shown) and the retrieval device 300 coupled to a distal end of the shaft. The retrieval device 300 may be movable between at least a closed state and an open state. For example, the retrieval device 300 may radially compress into the closed state when positioned within a sheath or lumen (not shown), and radially expand when positioned outside of the sheath or lumen. The shaft may be at least partially disposed in the sheath, such that the shaft and the sheath may move (e.g., translate and/or rotate) relative to each other. In the open state, the retrieval device 300 may define a cavity for capturing one or more objects therein (e.g., biliary stone from bile duct).

The retrieval device 300 may include a plurality of wires 302. The plurality of wires 302 may include proximal ends coupled to the distal end of the shaft positioned at a proximal end of the retrieval device 300 (not shown). As shown in the partially transparent view of FIG. 3, the plurality of wires 302 include distal ends 304 coupled to a distal tip 310 positioned at a distal end 306 of the retrieval device 300. The distal tip 310 may include a body 312 extending along a longitudinal axis between a proximal surface 314 and a distal surface 316 of the distal tip 310. The body 312 of the distal tip 310 may include an inner surface defining a lumen 308 extending therein from the proximal surface 314 toward the distal surface 316. The distal tip 310 may include, for example, a tube or a hypotube that is configured to receive distal ends 304 of the plurality of wires 302 therein. The distal ends 304 of wires 302 may be secured within the distal tip 310 using one or more fixation or coupling processes (e.g., crimping, welding, soldering, adhesive bonding, etc.). In one example, a proximal opening at the proximal surface 314 of the distal tip 310 may be shaped and dimensioned to receive the distal ends 304 of the plurality of wires 302 therein, and at least one of the distal ends 304 may be secured within the lumen 308 of the distal tip 310 using at least one fixation process.

In some aspects, it may be useful to couple the retrieval device 300 to one or more medical instruments, for example, to guide and/or orient the retrieval device 300 during medical procedures. For example, the distal tip 310 may include a slotted hypotube having one or more apertures extending therein (e.g., radially inward) into the lumen 308 of the distal tip 310 to, for example, receive the medical instrument(s) therein. As shown, the body 312 of the distal tip 310 may include an opening 318 positioned along a length of the body 312 between the proximal surface 314 and the distal surface 316 of the distal tip 310. In one example, the opening 318 of the distal tip 310 is shaped and dimensioned to receive a distal portion of a guidewire 320 therein, such that the guidewire 320 may be advanced distally or otherwise slidably positioned through the opening 318 into the lumen 308 and through the distal opening of the lumen 308 formed by distal surface 316 of the distal tip 310.

In some aspects, the retrieval device 300 may be atraumatic or have atraumatic surfaces which, for example, allow atraumatic insertion and/or movement of the distal tip 310 into a body lumen of a patient. In one example, the opening 318 of the distal tip 310 may have a smooth or curved edge, for example, to allow the guidewire 320 to move through and relative to the distal tip 310 without causing damage to the guidewire 320.

FIG. 4 shows a medical device, according to aspects of the disclosure. The medical device includes a retrieval device 400. Aspects of the retrieval device 400 may be similar to the retrieval device 100 (FIGS. 1A-1B), the retrieval device 200 (FIG. 2), and/or the retrieval device 300 (FIG. 3), the details of which are omitted for brevity. For instance, a medical device may include a shaft (not shown) and the retrieval device 400 coupled to a distal end of the shaft. The retrieval device 400 may be movable between at least a closed state and an open state. The retrieval device 400 may radially compress into the closed state and radially expand into the open state. For example, the retrieval device 400 may radially compress into the closed state when positioned within a sheath of the medical device, and radially expand when positioned outside of the sheath. The shaft may be at least partially disposed in the sheath, such that the shaft and the sheath may move (e.g., translate and/or rotate) relative to each other. In the open state, the retrieval device 400 may define a cavity for capturing one or more objects therein (e.g., biliary stone from bile duct).

The retrieval device 400 may include a plurality of wires 402 (hereinafter, “the wires 402”). The wires 402 may include proximal ends coupled to the distal end of the shaft positioned at a proximal end of the retrieval device 400 (not shown). The wires 402 may include distal ends coupled to a distal tip 410 positioned at a distal end 406 of the retrieval device 400. The wires 402 of the retrieval device 400 may be grouped into two or more sets or groupings of wires 402. For example, the wires 402 of the retrieval device 400 may be grouped into a first set 402A, a second set 402B, a third set 402C, and a fourth set 402D. Each set of the wires 402 may be respectively secured in one or more openings formed in the distal tip 410. In one example, the wires 402 of the retrieval device 400 may include eight wires, such that each of the first set 402A, the second set 402B, the third set 402C, and the fourth set 402D include two of the wires 402 having respective distal portions coupled together within respective openings 414 of the distal tip 410 to define respective sets. Although it should be understood that each set of the wires 402 may have more or less wires defining respective sets.

In some aspects, the distal tip 410 may include a body 412 extending along a longitudinal axis between a proximal surface and a distal surface of the distal tip 410. The body 412 of the distal tip 410 may include an inner surface defining a lumen 408 extending therein from the proximal surface toward the distal surface. The distal tip 410 may include, for example, a slotted hypotube that is configured to receive distal ends of the wires 402 therein, and which are secured within the distal tip 410 using one or more fixation processes. The distal tip 410 may include one or more openings extending through the body 412 into the lumen 408 and that are configured to receive one or more sets of the wires 402 therein. As shown, the body 412 of the distal tip 410 may include a plurality of openings 414 respectively extending therein into the lumen 408 of the distal tip 410. In one example, the plurality of openings 414 are positioned and spaced apart circumferentially on the body 412 relative to the longitudinal axis of the retrieval device 400, such that each of the plurality of openings 414 extends along a respective central axis that is oriented at a respective angle relative to the longitudinal axis and into the lumen 408 of the distal tip 410. The plurality of openings 414 may each have a shape and dimension to respectively receive one or more sets of the plurality of wires 402 therein.

As shown, the plurality of openings 414 of the distal tip 410 may include a first opening 414A, a second opening 414B, and a third opening 414C. In one example, the retrieval device 400 is assembled such that the first opening 414A receives the first set 402A of the wires 402 therein, the second opening 414B receives the second set 402B of the wires 402 therein, and the third opening 414C receives the third set 402C of the wires 402 therein. The plurality of openings 414 may include a fourth opening (not shown) that receives the fourth set 402D of wires 402 (e.g., positioned on the body 412 approximately 180 degrees away circumferentially from the second opening 414B relative to the longitudinal axis of the retrieval device 400). The wires 402 may converge in the lumen 408 of the distal tip 410 along the longitudinal axis and be secured or otherwise coupled to the body 412 therein.

In some aspects, it may be useful to couple the retrieval device 400 to one or more medical instruments, for example, to guide or orient the medical instrument(s) relative to the distal tip 410. As shown, the retrieval device 400 may include a guide insert 420. The guide insert 420 may be received within, or otherwise secured to, the distal tip 410 of the retrieval device 400. In one example, the guide insert 420 may be inserted into the lumen 408 through the distal end of the distal tip 410 and secured therein using one or more fixation or coupling processes (e.g., crimping, welding, soldering, adhesive bonding, etc.). In another example, the guide insert 420 may be secured to the distal tip 410 via compression fit, a snap fit, a friction fit, etc. In yet another example, the guide insert 420 may be secured to the distal tip 410 using one or more attachment features (not shown), such as external thread and internal thread. However, it should be understood that other processes or mechanisms may additionally or alternatively be used to secure the guide insert 420 and the distal tip 410.

In some aspects, the guide insert 420 may be shaped and dimensioned to guide and/or orient the medical instrument(s) relative to the distal tip 410. The guide insert 420 may include one or more slots configured to receive, guide, and/or orient the medical instrument(s) therein. As shown, the guide insert 420 includes a body 422 extending between a proximal end and a distal end of the guide insert 420. The body 422 may include an inner surface defining a guide slot 428 extending therein between a first aperture 424 and a second aperture 426 of the guide insert 420. The first aperture 424 and the second aperture 426 may each have a shape and dimension for receiving a distal portion of the medical instrument(s) therein. The medical instrument(s) may be advanced distally or otherwise slidably positioned through the first aperture 424 into the guide slot 428 and through the second aperture 426 of the guide insert 420, such that a distal portion of the medical instrument(s) extends distal of the distal end of the guide insert 420. The guide slot 428 of the guide insert 420 may restrict or constrain the orientation and/or the movement of the medical instrument(s) relative to the distal tip 410. In one example, the guide slot 428 of the guide insert 420 may receive a portion of a guidewire therein.

In some aspects, one or more openings of the distal tip 410 may at least partially align with one or more apertures of the guide insert 420 (e.g., in the open state of the retrieval device 400 during medical procedures). For example, the distal tip 410 may include an opening 416 that at least partially aligns with the first aperture 424 of the guide insert 420. In one example, a proximal end of the guide insert 420 is inserted into the lumen 408 of the distal tip 410, such that a central axis of the first aperture 424 of the guide insert 420 is aligned with a central axis of the opening 416 of the distal tip 410. The second aperture 426 at a distal end of the guide insert 420 may be at least partially aligned with a distal opening of the distal tip 410. During medical procedures, for example, the medical instrument (e.g., guidewire) may be slidably positioned through the opening 416 of the distal tip 410, and through the first aperture 424 into the guide slot 428 of the guide insert 420. The medical instrument may be positioned within or through the guide slot 428 and the second aperture 426 of the guide insert 420, such that a distal portion of the medical instrument is positioned distal of a distalmost end the guide insert 420. In one example, the distal portion of the medical instrument may be oriented at an angle (e.g., acute angle) relative to the longitudinal axis of the retrieval device 400. In another example, the guide slot 428 of the guide insert 420 is approximately parallel to the longitudinal axis of the retrieval device 400.

In some aspects, the guide insert 420 may be atraumatic or have atraumatic surfaces which, for example, allow atraumatic insertion of the retrieval device 400 into the body lumen of the patient. For instance, in some examples, the guide insert 420 has an outer surface 430 which is shaped and dimensioned to contact tissue of the patient without causing damage to said tissue, such as a distally tapering surface.

Each of the aforementioned devices, systems, and methods may be used for medical procedures to extract one or more objects from a patient. By providing a medical device capable of coupling a medical instrument to a retrieval device as discussed herein, known problems associated with extraction medical procedures and/or other aspects of invasive surgical procedures may be reduced or avoided. For example, providing a retrieval device that includes a distal tip coupled to the medical instrument and coupled to the plurality of wires of the retrieval device mitigates the need for additional coupling devices, which may break or cause issues during extraction medical procedures. Additionally, relative to conventional approaches, devices discussed herein may require less material to manufacture and request less time to assemble. Based on these aspects, physicians or other users of may reduce the overall procedure time, increase efficiency of procedures, and/or avoid unnecessary harm to a patient's body during extraction medical procedures. Furthermore, in some aspects, the devices, systems, and methods discussed herein may allow for a retrieval device to be movably coupled to one or more medical devices, for example, a guidewire, and the one or more medical devices may help the user deliver or position the retrieval device at or relative to a target treatment site.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device, comprising:
a sheath;
a shaft, wherein at least a portion of the shaft is movably disposed within the sheath; and
a retrieval device coupled to a distal end of the shaft, wherein the retrieval device is movable between at least a closed state and an open state, wherein the retrieval device is configured to radially compress into the closed state when positioned within the sheath, and wherein the retrieval device is configured to radially expand into the open state when positioned outside of the sheath, wherein the retrieval device comprises:
a plurality of wires coupled to the distal end of the shaft; and
a distal tip coupled to distal ends of the plurality of wires, wherein the distal tip comprises a body, and a channel extending through the body between a first opening and a second opening,
wherein the channel of the distal tip is configured to receive a medical instrument therein.

2. The medical device of claim 1, wherein the distal tip comprises a slot extending through the body along a longitudinal axis of the distal tip, and wherein the slot is coupled to distal ends of the plurality of wires therein.

3. The medical device of claim 2, further comprising a tube coupled to distal ends of the plurality of wires, and wherein the slot of the distal tip is coupled to the tube received therein.

4. The medical device of claim 3, wherein the plurality of wires is coupled within the tube via an adhesive, a crimp, a weld, or a combination thereof.

5. The medical device of claim 3, wherein the tube is coupled within the slot of the distal tip via an adhesive, a crimp, a weld, or a combination thereof.

6. The medical device of claim 3, wherein the tube comprises:
a body having a lumen extending therein along a longitudinal axis between a proximal end and a distal end of the distal tip; and
at least one opening extending through the body into the lumen,
wherein at least one of the plurality of wires is received through the at least one opening of the tube and secured within the lumen of the body.

7. The medical device of claim 6, wherein at least one opening of the tube comprises:
a plurality of openings extending through the body into the lumen and positioned circumferentially about the longitudinal axis, wherein each of the plurality of openings is configured to receive at least one of the plurality of wires therein.

8. The medical device of claim 2, wherein the plurality of wires is coupled to the slot of the distal tip via an adhesive, a crimp, a weld, or a combination thereof.

9. The medical device of claim 2, wherein the second opening of the channel is positioned distal of the slot relative to the longitudinal axis of the distal tip.

10. The medical device of claim 2, wherein the channel of the distal tip extends through the body along an axis that is oriented at an acute angle relative to the longitudinal axis of the distal tip.

11. The medical device of claim 10, wherein the body of the distal tip comprises a lip, wherein the lip is formed by a radially outward extension positioned proximal of a proximal end of the slot of the distal tip.

12. The medical device of claim 11, wherein a distal end of the sheath includes a distal surface configured to contact the lip of the distal tip in response to proximal translation of the retrieval device relative to the sheath.

13. The medical device of claim 1, wherein the medical instrument includes a guidewire slidably positioned in the channel of the distal tip.

14. The medical device of claim 1, wherein the distal tip includes a distally tapering outer surface at a distal end of the distal tip.

15. The medical device of claim 1, wherein the plurality of wires radially expands into the open state to define a cavity configured to capture an object therein.

16. A medical device comprising:
a sheath;
a shaft, wherein at least a portion of the shaft is movably disposed within the sheath; and
a retrieval device coupled to a distal end of the shaft, wherein the retrieval device is movable between at least a closed state and an open state, wherein the retrieval device is configured to radially compress into the closed state when positioned within the sheath, and wherein the retrieval device is configured to radially expand into the open state when positioned outside of the sheath, wherein the retrieval device comprises:
a plurality of wires, wherein proximal ends of the plurality of wires are coupled to the distal end of the shaft;
a distal tip including a body and a slot, wherein the slot extends through the body along a longitudinal axis of the distal tip, wherein the slot of the distal tip is configured to receive distal ends of the plurality of wires therein; and
a guide insert disposed in a distal portion of the slot of the distal tip, wherein the guide insert is configured to receive a portion of a guidewire.

17. The medical device of claim 16, wherein the guide insert comprises a body and a guide slot, wherein the guide slot extends through the body between a first aperture and a second aperture, wherein the guide slot is configured to receive the portion of the guidewire.

18. The medical device of claim 17, wherein the distal tip comprises:
a body having a lumen extending therein along a longitudinal axis between a proximal end and a distal end of the distal tip; and
a plurality of openings extending through the body into the lumen and positioned circumferentially about the longitudinal axis,
wherein at least one of the plurality of openings is configured to receive at least one set of the plurality of wires therein.

19. The medical device of claim 18, wherein the first aperture of the guide insert aligns with at least one of the plurality of openings of the distal tip to receive the portion of the guidewire therein.

20. A medical system, comprising:
a sheath;
a shaft, wherein at least a portion of the shaft is movably disposed within the sheath;
a retrieval device coupled to a distal end of the shaft, wherein the retrieval device is movable between at least a closed state and an open state, wherein the retrieval device is configured to radially compress into the closed state when positioned within the sheath, and wherein the retrieval device is configured to radially expand into the open state when positioned outside of the sheath, wherein the retrieval device comprises:
a plurality of wires, wherein proximal ends of the plurality of wires are coupled to the distal end of the shaft; and
a distal tip coupled to distal ends of the plurality of wires,
wherein the distal tip comprises a body, a slot extending through the body along a longitudinal axis of the distal tip, and a channel extending through the body along an axis that is oriented at a non-zero angle relative to the longitudinal axis, wherein the slot is coupled to distal ends of the plurality of wires therein; and
a guidewire movably disposed in the channel of the distal tip.
